(19) United States
(12) Patent Application Publication
(10) Pub. No.: US 2008/0097380 A1
(43) Pub. Date: Apr. 24, 2008

(54) METHOD AND APPARATUS FOR CREATING VARIABLE STIFFNESS AND/OR REDUCTION OF SOFT TISSUE

(76) Inventor: Kasey K. Li, Palo Alto, CA (US)

Correspondence Address: LEVINE BAGADE HAN LLP, 2483 EAST BAYSHORE ROAD, SUITE 100, PALO ALTO, CA, 94303, US

(21) Appl. No.: 11/534,196

(22) Filed: Sep. 21, 2006

(51) Int. Cl. A61M 5/32; A61M 31/00

(52) U.S. Cl. 604/506; 604/77

(57) ABSTRACT

A method and apparatus for creating variable stiffness along a soft tissue region is disclosed herein generally having an implantable device of bio-absorbable or biodegradable material. The implantable device has a drug of variable concentration along the length of the device to promote formation of an abscess and/or induce tissue necrosis following placement of the device in soft tissue. The abscess and/or tissue necrosis forms scar tissue having a variable stiffness along the soft tissue which in turn reduces the vibration of the soft tissue.

### BACKGROUND OF THE INVENTION

The present invention relates to apparatus and methods for creating variable stiffness of soft tissue within a patient. More particularly, the present invention relates to methods and apparatus for altering a region of soft tissue, e.g., soft palate, a portion of the tongue or base of the tongue, lateral pharyngeal wall, turbinate tissue, etc., to alter a physical characteristic of the soft tissue. For example, the soft tissue may be stiffened to alter its vibrating characteristics and/or to prevent or inhibit collapse of the tissue to prevent obstruction of an airway, e.g., for the treatment of snoring or sleep apnea.

When awake, the muscles of the throat typically hold the throat open, so that air passes in smoothly as we breathe. When asleep, these muscles typically relax and the area at the back of the throat sometimes narrows. The same amount of air passing through this smaller opening can cause pressure which results in the tissues surrounding the opening to vibrate. People who snore may have various reasons for the narrowing of the area at the back of the throat. The narrowing can be caused by tissues within or around the nose, mouth, or throat.

Snoring can occur when the roof of the mouth (soft palate and uvula), and sometimes the base of the tongue, starts to vibrate intermittently as a result of excessive pressure. The soft palate is typically the least supported of the tissue structures in the throat, and is therefore more prone to vibrate the other surrounding tissue areas.

Snoring may also occur if insufficient air is taken in by nasal breathing. Nasal obstruction, or a stuffy nose, is often caused by an enlargement of the nasal turbinates. When nasal congestion forces mouth breathing during sleep, greater negative pressure develops behind the uvula and soft palate. This negative pressure increases the vibration of the uvula and soft palate which in turn creates the sound of snoring.

Snoring is a common symptom of obstructive sleep apnea although snoring by itself does not involve the cessation of breathing. Obstructive sleep apnea is a breathing obstruction which requires the sleeper to awaken to begin breathing again. A person with sleep apnea typically awakes several times a night to regain breathing.

Severe snoring may additionally causes sleep deprivation for both the snorer and the sleeping partner. Some common effects of sleep deprivation may include daytime sleepiness, a compromised immune system, poor mental health, decreased productivity, low energy, etc. Moreover, conditions such as snoring and/or sleep apnea may not only cause drops in the blood oxygenation level, but may also adversely affect the heart by increasing blood pressure and pulse rate. Patients often complain of a sore throat and nearly always complain of a dry throat. This is typically caused by the soft palate vibrating for several hours leading to trauma of the tissues with inflammation and often edema of the palate and uvula.

The elimination of snoring and the various degrees of sleep apnea have been attempted because of their various effects on the body. A number of conventional treatments exist such as mask and nasal continuous positive airway pressure (CPAP) devices but compliance is poor. There are also several surgical treatments such as Uvulopalatopharyngoplasty (UPPP) and Thermal Ablation Palatoplasty (TAP) which are painful and often have unpleasant side effects.

Palate implants are also used to treat snoring and typically result in less tissue inflammation and less pain. However, many patients may have an aversion to having an implanted device remain in a part of their body.

Accordingly, there is a need for a system which maintains the patency of a person's airways during sleep without painful side effects, long recovery periods, and/or the presence of a foreign object implanted permanently in the body.

### BRIEF SUMMARY OF THE INVENTION

An apparatus for creating variable stiffness along a soft tissue region may generally comprise, in one variation, an implantable device of bio-absorbable or biodegradable material where the device has at least one drug to promote formation of an abscess following placement of the device in soft tissue. The drug may be of a variable concentration gradient along a length of the device whereby the abscess forms variable degrees or amounts of scar tissue along the length of the device thereby having variable stiffness along the soft tissue region. Additionally and/or alternatively, aside from abscess formation, the drug may induce tissue necrosis following placement of the device in soft tissue. The tissue necrosis leads to variable degrees or amounts of scar tissue formation and retraction of a portion of the soft tissue, ultimately resulting in an overall reduction of volume of the soft tissue region. Reduction of tissue volume may be an alternative mechanism for creating variable stiffness along the soft tissue region.

The implantable device may be a gel mixture and/or alternatively a flexible structure having any number of various structure configurations. One variation may include a structure having some geometric shape or configuration, such as tubes, spheres, ovals, rectangles, cylinders, and tapered. Another variation may include a structure that comprises a plurality of intertwined fibers. Another variation may also include a structure that has at least a portion of the device that is fibrous.

Additionally, the implantable device may have an outer surface that is smooth or substantially smooth. Alternatively, the implantable device may also have an outer surface that is rough or substantially rough.

In one variation, the drug concentration gradient along the length of the device may be linearly increasing or decreasing relative to its length. In another variation, the drug concentration gradient along the length of the device may be non-linearly or non-uniformly concentrated. In yet another variation, the drug concentration gradient along the length of the device may be concentrated in a stepped manner.

When initially implanted or placed into the soft tissue region to be treated, the implantable device may be inserted in a portion of a soft palate, tongue, and/or turbinate. When inserted, the implantable device may be positioned in any number of positions relative to the soft tissue region; for instance, the device may be aligned anterior to posterior of the soft tissue region. A second variation includes aligning the implantable device at an angle relative to an anterior-to-posterior axis of the soft tissue. Yet another variation includes aligning the implantable device transversely to the anterior-to-posterior axis of the soft tissue.

Furthermore, a plurality of implantable devices may be inserted into the soft tissue in any number of positional configurations. For instance, the implantable devices may be inserted parallel or at an angle relative to one another within the tissue. Additionally, the implantable devices may also have the same or different drug concentration gradient relative to one another.

### BRIEF DESCRIPTION OF THE SEVERAL VIEWS OF THE DRAWINGS

FIG. 1 shows a partial cross-sectional view of a patient's oral and nasal cavity illustrating a position of the patient's soft palate and tongue with air passing normally through the cavities.

FIG. 2 shows the tongue in its relaxed and collapsed state, for example when the patient is asleep, illustrating the base of the tongue resting directly against the soft palate and also obstructing the airway passage.

FIG. 3 shows the soft palate in a relaxed and collapsed state illustrating the soft palate resting against the nasopharynx and obstructing the airway passage.

FIG. 4A illustrates a side view of the soft palate having a variation of an implantable device placed therein.

FIG. 4B is a front view of an interior of a patient's mouth illustrating the soft palate containing an implantable device as shown in FIG. 4A.

FIGS. 5A and 5B show perspective and end views, respectively, of a variation of the implantable device shown in FIGS. 4A-4B having a cylindrical shape.

FIGS. 6A and 6B show perspective and end views, respectively, of another variation of the implantable device having an elliptical cross-sectional shape.

FIGS. 7A and 7B show perspective and end views, respectively, of another variation of the implantable device having a rectangular cross-sectional shape.

FIGS. 8A and 8B show perspective and end views, respectively, of another variation of the implantable device shown having a tapered shape (i.e. conical).

FIGS. 9A and 9B show perspective and end views, respectively, of another variation of the implantable device having a braided configuration.

FIGS. 10A and 10B show side and end views, respectively, of another variation of the implantable device having a smooth first portion and a fibrous second portion.

FIG. 11 shows a perspective view of the implantable device shown in FIG. 5A and having a fibrous surface.

FIGS. 12A and 12B illustrate side and front views, respectively of an abscess formed within a soft palate of the patient and the formation of a variable amount of scar tissue.

FIGS. 13 and 14 are graphs schematically illustrating variable drug concentrations levels which may be concentrated along a length of an implantable device.

FIG. 15 shows a variation for positioning the implantable device in an alternate transverse orientation within a soft palate of a patient.

FIG. 16 shows another variation for positioning the implantable device in an alternate angled orientation within a soft palate of a patient.

FIG. 17 shows yet another variation for positioning multiple implantable devices within a soft palate of a patient.

FIG. 18 shows yet another variation for positioning multiple implantable devices in an angled orientation relative to one another within a soft palate of a patient.

FIG. 19 shows a partial cross-sectional view of a patient's oral and nasal cavity illustrating an implantable device within the base of the tongue.

FIG. 20 shows a partial cross-sectional view of a patient's oral and nasal cavity illustrating an implantable device within the inferior turbinate.

FIG. 21 shows a variation of a delivery tool for delivery of the implantable device.

FIG. 22 shows a cross-sectional side view of a distal tip of the delivery tool to reveal at least one implantable device loaded into the delivery lumen of the tool.

FIG. 23 is a cross-sectional side view of the soft palate showing a palatal muscle in the soft palate.

FIG. 24 illustrates the delivery tool of FIG. 21 advanced into the soft palate.

FIG. 25 illustrates the implantable device having been deployed into a portion of the soft palate and removal of the delivery tool.

### DETAILED DESCRIPTION OF THE INVENTION

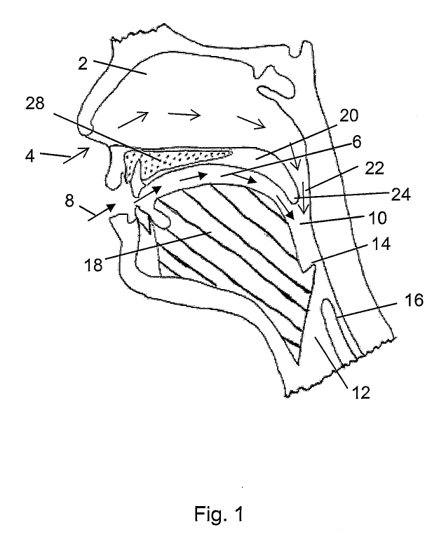

As illustrated in FIG. 1, air is illustrated flowing through the nasal cavity (nose) 2 as shown by arrows 4 as well as through the oral cavity (mouth) 6, as shown by arrows 8. The incoming air is passed through the nose 2 and/or mouth 6 and into the pharynx (throat) 10, where it then passes downward to the trachea 12. The epiglottis 14 is a flap of tissue that guards the entrance to the trachea 12, closing when solids or fluids are swallowed in order to prevent food or fluid from entering the lungs. The esophagus 16 is the passage leading from the mouth 6 and throat 10 to the stomach. The trachea 12 is the passage leading from the pharynx 10 to the lungs.

Snoring occurs when air fails to flow smoothly through the nose 2 or the mouth 6 and/or when the soft tissues or muscles in the air passages vibrate at some resonate frequency. As a person falls asleep, the muscles in the tongue 18, throat 10 and soft palate 20 relax. As shown in FIG. 2, this muscle relaxation causes the throat tissues to sag where the tongue 18 and upper throat (nasopharynx) 22 contact the soft palate 20 and/or uvula 24, which begins to vibrate as air passes between them. As illustrated in FIG. 3, the soft palate 20 may also sag toward the nasopharynx 22, narrowing the airway. The narrower the airway becomes, the greater the vibration and the more intense the snoring.

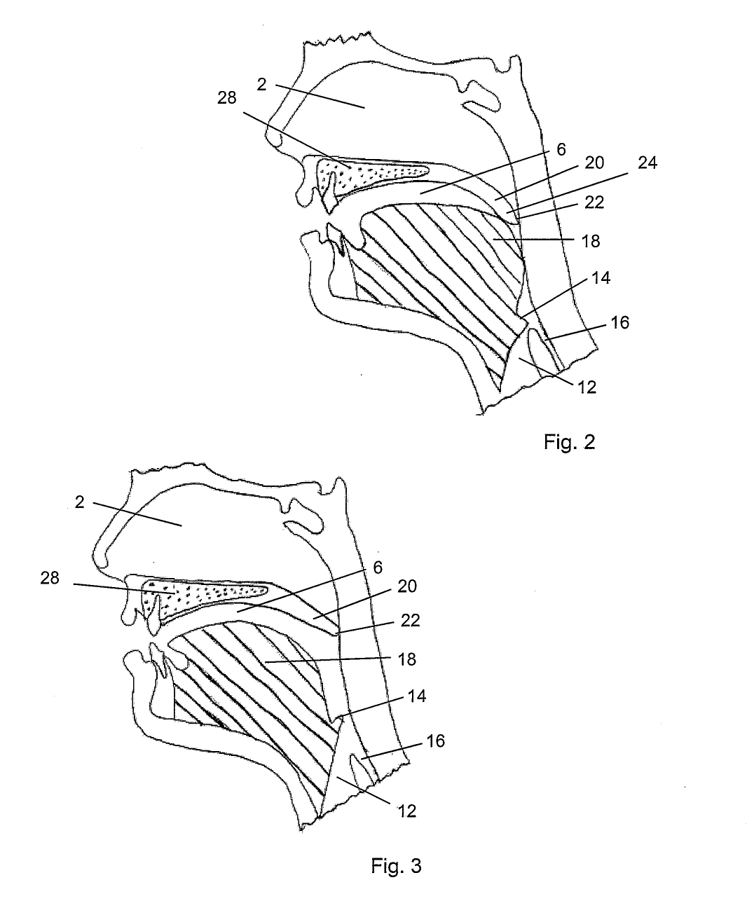

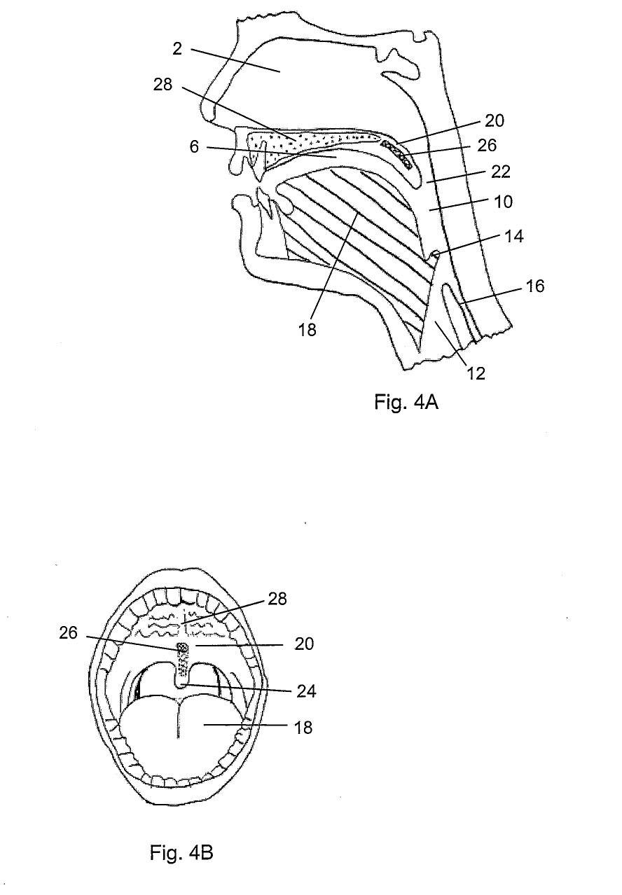

The soft palate 20 is not structurally supported by bone or hard cartilage and is therefore more prone to vibrate. Altering the mass and/or stiffness of the soft palate 20 may reduce the vibration level and in turn decrease the intensity of snoring. FIGS. 4A-4B illustrate a variation in which at least one implantable device 26, described in further detail below, may be at least partially or fully embedded, implanted, or otherwise inserted in the soft palate 20 in close proximity to the hard palate 28. In inserting the implantable device 26 in the soft palate 20, the mass and/or stiffness of the soft palate 20 is increased, which in turn decreases the vibration of the soft palate 20. Aside from acting as a structural enhancement, the implantable device 26 may be made of a biocompatible material which is biodegradable. Accordingly, the implantable device 26 may be configured to initially provide a structural support to the soft palate and eventually degrade or absorb completely or partially into the body, thereby leaving a stiffening region of scar tissue which may continue to naturally provide a structural support to alter the vibrational frequency of the soft tissue, as described in further detail below.

Biodegradable materials may include materials that are bioabsorbable, bioreabsorbable, or that degrade over time and are metabolized by the body. Some examples of biodegradable materials include, but are not limited to, absorbable hemostat such as microfibrillar collagen, absorbable gelatin sponge, polyglycolic acid (PGA), poly-L-glycolic acid (PLGA), polylactic acid (PLA), polycaprolactone (PCL), polyethyl acrylate (PEA), polydioxanone (PDS), and combinations of polymer of lactic acid and glycolic acid, etc. The implantable device 26 may degrade, for example, anywhere from 1-2 weeks, but may degrade over a longer or shorter period of time, depending upon the desired results.

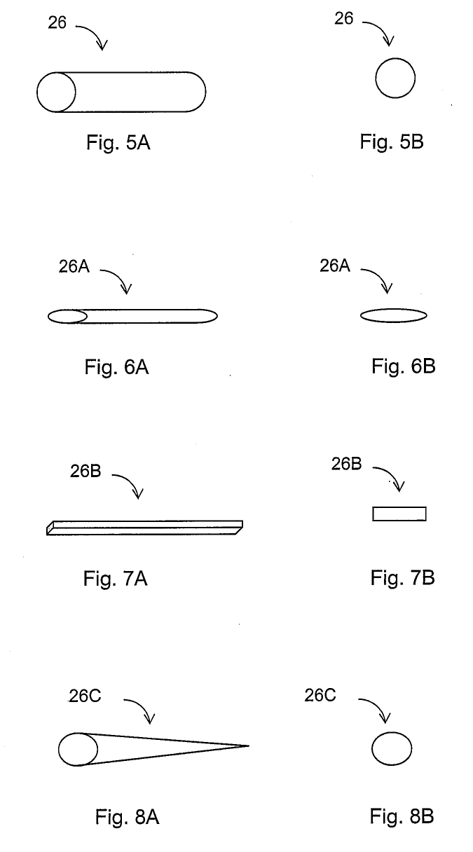

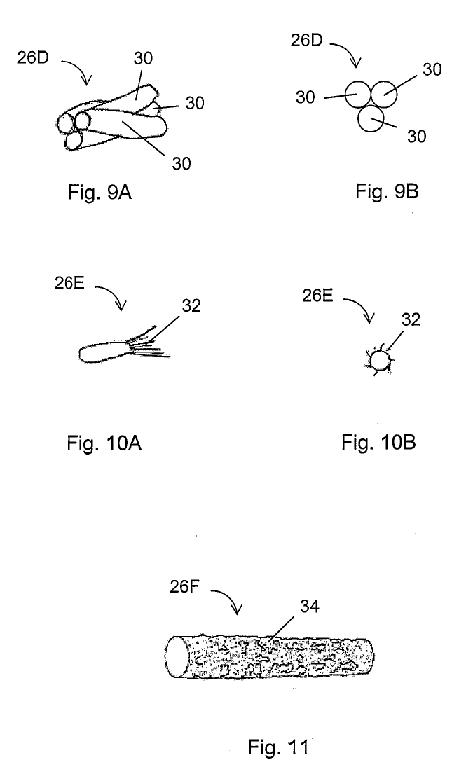

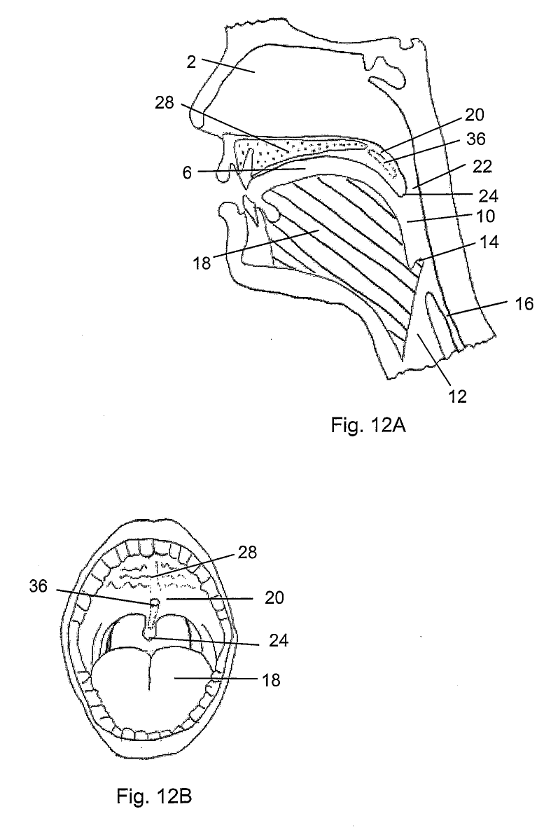

The implantable device 26 may be configured into a variety of geometric shapes, as illustrated in FIGS. 5A-8B. Some non-limiting examples include tubes or cylinders 26, as shown in the perspective and end views of FIGS. 5A and 5B, elliptical cross-sectional shapes 26A as shown in the perspective and end views of FIGS. 6A and 6B, rectangular cross-sectional shapes 26B as shown in the perspective and end views of FIGS. 7A and 7B, tapered shapes 26C as shown in the perspective and end views of FIGS. 8A and 8B. Additional shape configurations may also include spheres, ovals, or any other shapes, as practicable. Another variation includes a device 26D having two or more intertwined fibers 30, as shown in the partial perspective and end views, respectively, of FIGS. 9A-9B. In this variation, the individual fibers 30 may be intertwined randomly or in a uniform pattern, much like a braid or rope. FIGS. 10A-10B demonstrate yet another variation where the implantable device 26E has a portion of the length that is fibrous 32 extending from a non-fibrous portion. These shapes are for illustrative purposes and are not intended to be limiting.

The implantable device 26 is ideally sized for insertion into a soft tissue region and accordingly may have a length of, e.g. between 2-30 mm, a width of, e.g. between 2-5 mm, and a depth of, e.g. between 2-4 mm. The size may vary depending on the deployment site in the soft tissue or the shape of the implantable device 26.

The outer surface of the implantable device 26 may be smooth or substantially smooth (e.g., the majority of the outer surface has an even consistency with minor irregularities) to potentially allow for ease of insertion into the soft tissue. Another variation, as shown in the device 26F in FIG. 1, includes an outer surface that may be rough 34 or substantially rough (e.g., the majority of the outer surface has an uneven consistency marked by irregularities) such that a rough surface may assist with anchoring the implantable device 26 and prevent migration after insertion.

The implantable device 26 may be filled, coated, layered and/or otherwise made with and/or from a drug known to one having ordinary skill in the art to induce tissue necrosis and/or promote the formation of an abscess 36. Abscesses 36 may occur when white blood cells gather in response to an infection (septic) or irritant (sterile), such as foreign bodies or drugs that have not been fully absorbed. The white blood cells produce oxidants (for example, superoxide radical) and enzymes to digest the irritant. The irritant is then broken down by the white blood cells into small pieces that can be transported through the bloodstream and eliminated from the body. The enzymes may also digest part of the body's tissues along with the irritant. The resulting liquid of this digestion is pus, which contains the remains of the irritant, tissue, white blood cells, and enzymes. Abscesses 36 caused by an irritant often heal into hardened scar tissue and this hardened scar tissue adds mass and stiffness to the soft tissue, as well as reduces the tissue volume. Some examples of the drug include, but are not limited to, alpha hydroxyl acid (such as glycolic acid, lactic acid, and citric acid), beta hydroxyl acid (such as salicylic acid), acetyl salicylic acid (ASA), doxycycline, bleomycin, aspirin, and ibuprofen. After the implantable device 26 is inserted into the deployment site within the soft tissue, the implantable device 26 begins to degrade and is metabolized by the body over time while the drug irritates the surrounding tissues and ideally forms an abscess 36 around the tissue contacted by the device. The abscess 36 may, for example, form 3-4 mm out around the implantable device 26. FIGS. 12A-12B illustrate the formation of an abscess 36 in the soft palate 20.

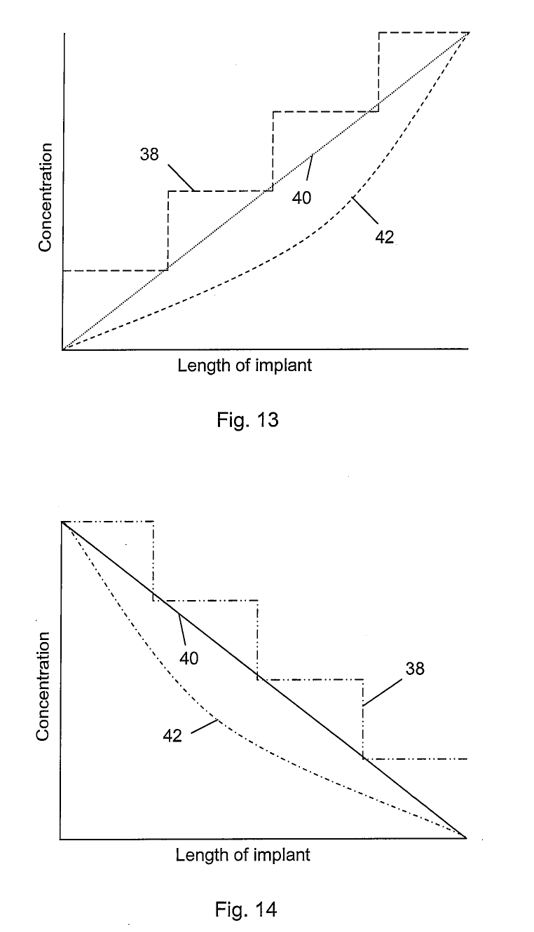

The drug may be concentrated along the length of the implantable device 26 in any number of concentration levels relative to the length, as illustrated in the graphs in FIGS. 13-14. Varying the concentration of the drug on the implantable device 26 may provide one method for controlling the degree of tissue necrosis and/or the size of the resulting abscess and ultimately for controlling the stiffness of the resulting scar tissue. For instance, the concentration may be increased along the length of the implantable device 26 as demonstrated in FIG. 13. The increase in concentration may be stepped 38, linear 40, or non-linear 42, or any combination thereof. Therefore, the resulting abscess may form around the device in a size and configuration which correlates to the concentration of the drug along the device. Moreover, the amount of scar tissue which forms in the abscess may thereby result in a mass of tissue which has a stiffness correlating to the amount of scar tissue formed along the length of the device.

Accordingly, a device having an increasing concentration of drug along its length may form an abscess around it which also increases in size according to the concentration of the drug. The resulting scar tissue may also accordingly form in increasing amounts within and around the abscess to thereby result in a scar tissue formation having stiffness which varies along a length of the originally implanted device, which is ultimately absorbed or degraded by the body leaving the scar tissue behind. Likewise, the concentration may also decrease along the length of the implantable device 26 as demonstrated in FIG. 14. The decrease in concentration may be likewise stepped 38, linear 40, or non-linear 42, or any combination thereof. The concentration may also be uniformly constant along the length of the implantable device 26.

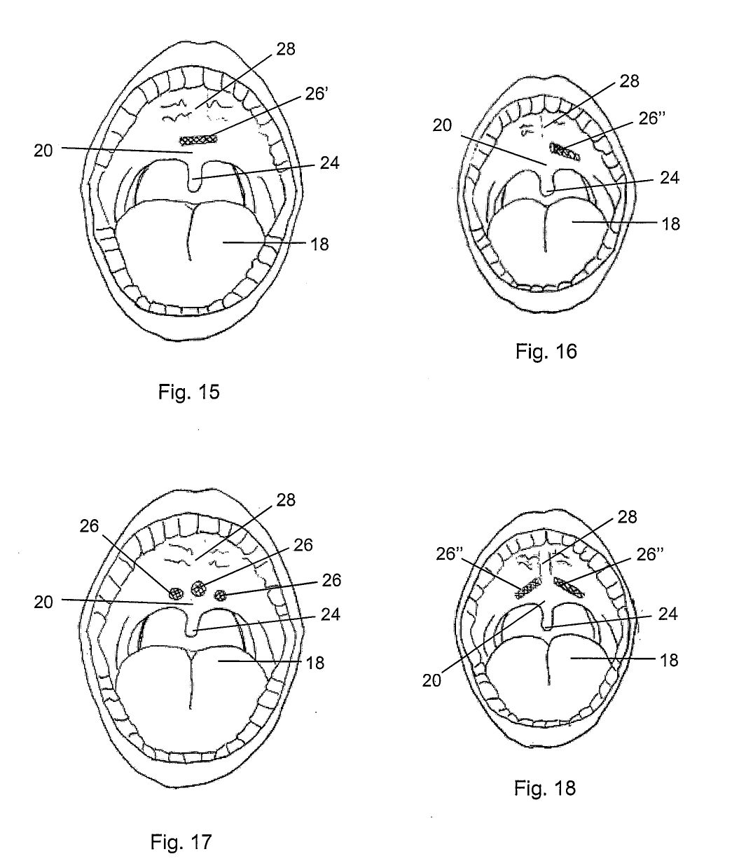

The implantable device 26 may be inserted into soft tissue in many different configurations. FIGS. 4A-4B show one variation where the implantable device 26 is aligned anterior to posterior within the soft palate 20. FIG. 15 demonstrates another variation where the implantable device 26′ is aligned transverse relative to the anterior to posterior axis of the soft palate 20. The implantable device 26″ may also be aligned at an angle relative to the anterior to posterior axis of the soft palate 20, as shown in FIG. 16.

Aside from orientation, the number of devices may also be varied depending upon the desired stiffness of the soft tissue. For instance, a plurality of implantable devices 26, i.e., at least two or more, may be inserted into the soft tissue. FIGS. 17-18 illustrate the insertion of more than one implantable device 26 into the soft palate. In a first variation, the implantable devices 26 may be oriented parallel relative to one another within the soft tissue, as shown in FIG. 17. Alternatively, the implantable devices 26″ may also be oriented at an angle relative to one another, as shown in FIG. 18. Moreover, the implantable devices 26 may be oriented in various combinations relative to one another; for instance, one or more devices may be parallel to one another while another device is angled.

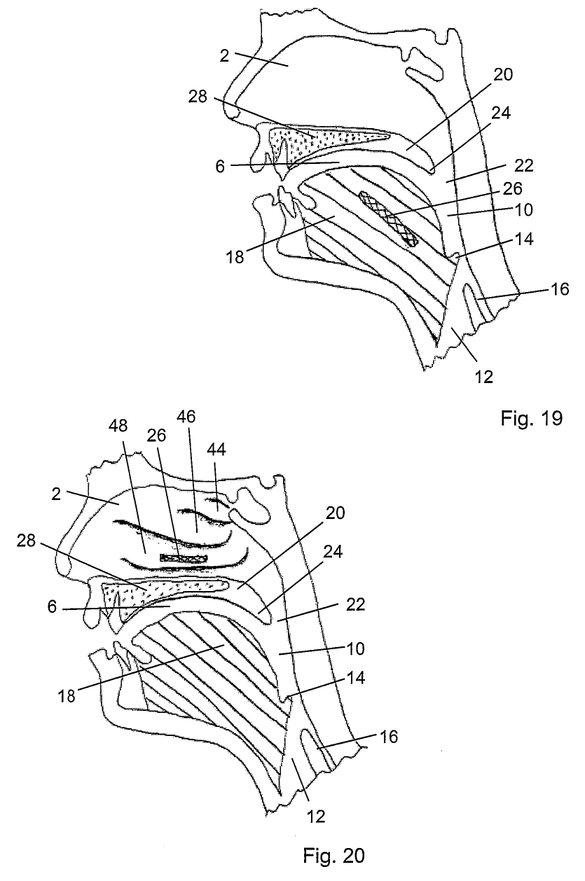

Aside from implantation into the soft palate, other tissue regions may be utilized such as those illustrated in FIGS. 19-20, which show alternative soft tissue insertion sites. FIG. 19 shows at least one implantable device 26 inserted into and/or near the base of the tongue 18. “Tongue snoring” is caused by the space behind the tongue 18 being restricted causing vibration as the air flows past. It is less intense than palatal snoring but is more likely to lead to obstruction to the flow of air, or sleep apnea. The implantable device 26 may also increase stiffness to the tongue 18 and may prevent the tongue 18 from sagging into the throat 10. Adding stiffness to the tongue 18 may also lower the vibration intensity. Variations of the implantable device 26 and methods described herein for soft tissue and/or the soft palate 20 may also apply to the implantable device 26 and methods for the tongue 18.

In addition to or in place of the tongue 18, the implantable device 26 may be inserted into-the soft tissue of the nasal cavity 2 (i.e., any one or all of the superior 44, middle 46, and/or inferior 48 turbinates). FIG. 20 illustrates the implantable device 26 inserted into a portion of the inferior turbinate 48, although the implantable device 26 may be inserted into any soft tissue within the nasal cavity 2. In this variation, the implantable device 26 may be inserted into the turbinate to widen the nasal passage and/or inhibit or decrease the amount of inflammation the turbinate tissue may undergo. As the implantable device 26 degrades, an abscess 36 may be formed within the turbinate tissue thereby creating scar tissue. The scar tissue may shrink the turbinate, resulting in a widened nasal passage. Variations of the implantable device 26 and methods described herein for soft tissue and/or the soft palate 20 also apply to the implantable device 26 and methods for the turbinate 18.

When more than one implantable device 26 is inserted into the soft tissue, the drug concentration gradient may vary relative to other implantable devices 26. One variation includes at least one implantable device 26 that has a drug concentration gradient that is stepped 38 combined with at least one other implantable device 26 that is linear 40. Another variation includes at least one implantable device 26 having a drug concentration gradient that is linear 40 combined with at least one other implantable device 26 that is non-linear 42. A third variation includes at least one implantable device 26 having a drug concentration gradient that is non-linear 42 combined with at least one other implantable device 26 having a drug concentration gradient that is stepped 38. Another variation includes any combination of drug concentration gradients (stepped 38, linear 40, and/or non-linear 42) on a plurality of implantable devices 26. The implantable devices 26 may also have a drug concentration gradient that is the same relative to other implantable devices 26.

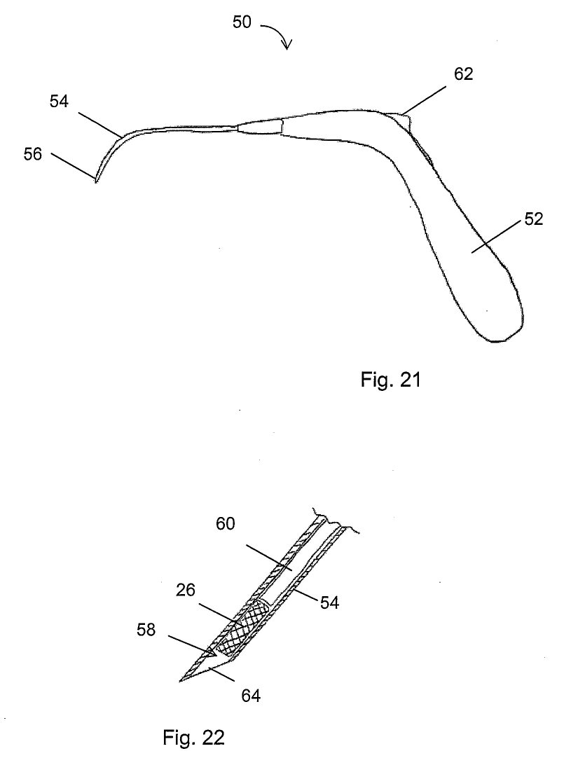

The implantable device 26 may be delivered to the deployment site within the soft tissue using any deployment tool 50. FIG. 21 illustrates an example of a deployment tool 50 which may be used to insert the implantable device 26 into the soft tissue. The deployment tool 50 has a handle 52 connected to a proximal end of a needle 54. As shown in FIG. 21, the needle 54 may have a distal portion which is curved relative to a length of the needle 54 to facilitate penetration and insertion into portions of the soft palate or other regions of tissue and a distal tip 56 that is shaped to penetrate soft tissue. The needle 54 defines a central lumen 58 within which one or more implantable devices 26 may be inserted, as shown in the cross-sectional view of FIG. 22. Proximally adjacent to the implantable device 26 is a plunger 60 slidably positioned within the lumen 58 such that a proximal portion of the plunger 60 is connected, coupled, or otherwise in communication with an actuator 62 located on the handle 52. The actuator 62 may comprise any number of actuation mechanisms and may be used to control the advancement or retraction of the plunger 60 relative to the needle 54 to eject or retract the implantable device 26. In one variation, the actuator 62 is in communication with the needle 54 and may proximally retract the needle 54 at least partially into the handle 52 while maintaining the plunger 60 stationary. When the needle 60 is urged proximally relative to the handle 52 via the actuator 62, the implantable device 26 is ejected from the opening 64 at the distal tip 56 of the needle 54 into the deployment site.

In another variation, the actuator 62 is in communication with the plunger 60 and the needle 54 is stationary. When the plunger 60 is urged distally relative to the handle 52 via the actuator 62, the implantable device 26 is ejected from the opening 64 at the distal tip 56 of the needle 54 into the deployment site. Using this deployment method, the implantable device 26 may be sized and shaped in such a manner to displace the soft tissue during insertion. For example, a cone shaped implantable device 26 as shown in FIGS. 8A-8B where the distal end is tapered, may facilitate entry into the soft tissue, although any shaped implantable device 26 may be used.

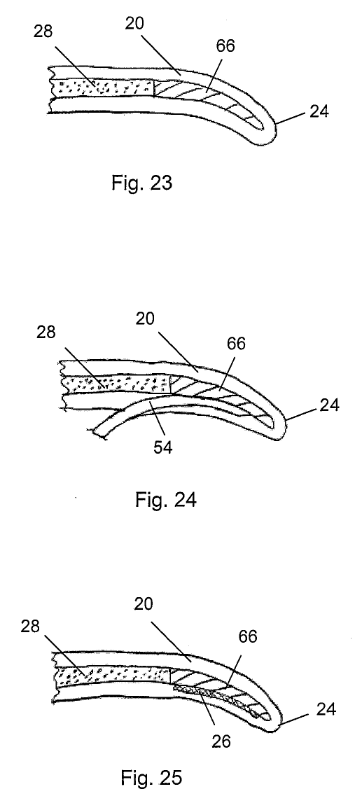

The deployment site of the implantable device 26 may be adjacent to an anatomical structure that acts as a hinge or a point of support, or the fulcrum. FIGS. 23-25 illustrate the deployment site of the implantable device 26 in the soft palate. FIG. 23 shows a side sectional view of the hard palate 28 and soft palate 20. Within the soft palate 20, there is a palatal muscle 66. The interface between the hard palate 28 and the palatal muscle 66 may function as a fulcrum for the soft palate 20. The distal tip 56 of the needle 54 of the deployment tool 50 is inserted into the soft palate 20 from the oral cavity 6 as shown in FIG. 24. The implantable device 26 is ejected from the distal tip 56 of the needle 54 and the needle 54 is then withdrawn, as shown in FIG. 25. The implantable device 26 may be inserted into the tongue 18 using the same method as the soft palate 20. A rhinoscope or other visualization tool known in the art may be utilized to assist with identifying the deployment site for this application or any other application as described herein, e.g., deployment within the turbinate.

While illustrative examples are described above, it will be apparent to one skilled in the art that various changes and modifications may be made therein. Moreover, various apparatus or methods described above are also intended to be utilized in combination with one another, as practicable. The appended claims are intended to cover all such changes and modifications that fall within the true spirit and scope of the invention.

What is claimed is:

1. An apparatus for creating variable stiffness along a soft tissue region comprising:
an implantable device of bio-absorbable or biodegradable material;
wherein the device has a drug to promote formation of an abscess and/or induce tissue necrosis following placement of the device in soft tissue; and
wherein the drug is of a variable concentration gradient along a length of the device whereby the abscess and/or tissue necrosis forms scar tissue having variable stiffness along the soft tissue region.

2. The apparatus of claim 1, wherein the soft tissue is a portion of a soft palate.

3. The apparatus of claim 1, wherein the soft tissue is a portion of a tongue.

4. The apparatus of claim 1, wherein the soft tissue is a portion of a turbinate.

5. The apparatus of claim 1, wherein the drug concentration gradient is linear.

6. The apparatus of claim 1, wherein the drug concentration gradient is non-linear.

7. The apparatus of claim 1, wherein the implantable device is flexible along the length.

8. The apparatus of claim 1, wherein the implantable device is a geometric shape selected from the group consisting of tubes, spheres, ovals, rectangles, cylinders, and tapered.

9. The apparatus of claim 1, wherein the implantable device comprises a plurality of intertwined fibers.

10. The apparatus of claim 1, wherein at least a portion of the implantable device is fibrous.

11. The apparatus of claim 1, wherein the implantable device has a smooth or substantially smooth outer surface.

12. The apparatus of claim 1, wherein the implantable device has a rough or substantially rough outer surface.

13. A method for creating variable stiffness of a soft tissue region comprising:
providing an implantable device of bio-absorbable or biodegradable material wherein the device has a drug of a variable concentration gradient along a length of the device; and
inserting the device into a desired deployment site within the soft tissue region.

14. The method of claim 13, wherein the soft tissue is a portion of a soft palate.

15. The method of claim 13, wherein the soft tissue is a portion of a tongue.

16. The method of claim 13, wherein the soft tissue is a portion of a turbinate.

17. The method of claim 13, wherein the drug concentration gradient is linear.

18. The method of claim 13, wherein the drug concentration gradient is non-linear.

19. The method of claim 13, wherein inserting comprises inserting the implantable device aligned anterior to posterior of the soft tissue region.

20. The method of claim 13, wherein inserting comprises inserting the implantable device at an angle relative to the anterior to posterior axis of the soft tissue.

21. The method of claim 13, wherein inserting comprises inserting the implantable device aligned transverse to the anterior to posterior axis of the soft tissue.

22. The method of claim 13, further comprising inserting a plurality of implantable devices.

23. The method of claim 22, wherein inserting comprises inserting the implantable device parallel relative to other implantable devices.

24. The method of claim 22, wherein inserting comprises inserting at least on implantable device at an angle relative to other implantable devices.

25. The method of claim 22, wherein inserting comprises inserting the implantable devices having the same drug concentration gradient relative to other implantable devices.

26. The method of claim 22, wherein inserting comprises inserting at least one implantable device having a different drug concentration relative to the other implantable devices.

27. The method of claim 13, wherein inserting comprises inserting the implantable device by placing the implantable device into a lumen of a needle and the needle is advanced through the soft tissue to the desired deployment site.

28. The method of claim 27, wherein the needle has a proximal end and a distal tip shaped for penetrating soft tissue.

29. The method of claim 27, wherein the lumen has a plunger adjacent to the implantable device.

30. The method of claim 29, wherein inserting the implantable device comprises ejecting the implantable device from the distal tip of the needle when the plunger is urged distally relative to the needle.

31. The method of claim 29, wherein the needle is slidable proximally relative to the plunger and the implantable device.

32. The method of claim 31, wherein inserting the implantable device comprises ejecting the implantable device from the distal tip of the needle when the needle is urged proximally relative to the plunger and the implantable device.

33. A system for creating variable stiffness of a soft tissue region comprising:
an implantable device of bio-absorbable or biodegradable material wherein the implantable device has a drug of a variable concentration gradient along a length of the implantable device; and
a delivery tool for inserting the implantable device into soft tissue.

34. The system of claim 33, wherein the soft tissue is a portion of a soft palate.

35. The system of claim 33, wherein the soft tissue is a portion of a tongue.

36. The system of claim 33, wherein the soft tissue is a portion of a turbinate.

37. The system of claim 33, wherein the drug concentration gradient is linear.

38. The system of claim 33, wherein the drug concentration gradient is non-linear.

39. The system of claim 33, wherein the implantable device is flexible along the length.

40. The system of claim 33, wherein the implantable device is a geometric shape selected from the group consisting of tubes, spheres, ovals, rectangles, cylinders, and tapered.

41. The system of claim 33, wherein the implantable device comprises a plurality of intertwined fibers.

42. The system of claim 33, wherein at least a portion of the implantable device is fibrous.

43. The system of claim 33, wherein the implantable device has a smooth or substantially smooth outer surface.

44. The system of claim 33, wherein the implantable device has a rough or substantially rough outer surface.

45. The system of claim 33, further comprising a plurality of implantable devices.

46. The system of claim 45, wherein the implantable devices have the same drug concentration gradient.

47. The system of claim 45, wherein at least one implantable device has a different drug concentration relative to the other implantable devices.

48. The system of claim 33, wherein the delivery tool comprises:
a handle;
a needle with a proximal end connected to the handle and a distal tip shaped for penetrating soft tissue wherein the needle defines a central lumen;
a plunger within the lumen adjacent to the implantable device; and
an actuator which controls the advancement or retraction of the implantable device.

49. The system of claim 48, wherein the actuator is connected to the plunger such that the implantable device is ejected from the distal tip of the needle when the plunger is urged via the actuator distally relative to the handle.

50. The system of claim 48, wherein the needle is slidable proximally relative to the handle.

51. The system of claim 50, wherein the actuator is connected to the needle such that the implantable device is ejected from the distal tip of the needle when the needle is urged via the actuator proximally relative to the handle.
